(19) United States
(12) Patent Application Publication
(10) Pub. No.: US 2024/0415689 A1
(43) Pub. Date: Dec. 19, 2024

(54) PAD-SHAPED FEMALE EXTERNAL CATHETER AND RELATED SYSTEMS AND METHODS

(71) Applicant: C.R. BARD, INC., Murray Hill, NJ (US)

(72) Inventors: Rebecca Weiss, Atlanta, GA (US); Jill W. Jones, Avondale Estates, GA (US); Jonathan Robichaud, Decatur, GA (US); Sharon D. West, Norristown, PA (US); Christopher J. Murray, Philadelphia, PA (US); Gabrielle Zingalis, Murray Hill, NJ (US); Mary Arwen Broughton, Cary, NC (US); Melody Mei Hee Kuroda, Durham, NC (US)

(21) Appl. No.: 18/335,579

(22) Filed: Jun. 15, 2023

(51) Int. Cl. A61F 5/44; A61F 5/455

(52) U.S. Cl. CPC A61F 5/4401; A61F 5/4556

(57) ABSTRACT

Embodiments disclosed herein are fluid collection devices, and related systems and methods of use and manufacture. The fluid collection device may include a fluid impermeable barrier, a porous material, and a conduit. The fluid impermeable barrier may have an elongated shape with a proximal region and a distal region, and including a first impermeable layer and a second impermeable layer opposite to the first impermeable layer and defining an opening. The first impermeable layer and the second impermeable layer at least partially defining a chamber therebetween, the chamber including a pocket portion that extends distally from the opening between the first impermeable layer and the second impermeable layer. The porous material extends across the opening and is positioned in the pocket portion of the chamber. The porous material is configured to space the first impermeable layer from the second impermeable layer in the pocket portion of the chamber.

### CROSS-REFERENCE TO RELATED APPLICATIONS

This application claims priority to U.S. Provisional Patent Application No. 63/633,026 filed on Jun. 21, 2022, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans, urinary catheters, adult diapers, and/or incontinence pads can be used to address some of these circumstances. However, each of these have several problems associated therewith. For example, bed pans can be prone to discomfort, pressure ulcers spills, and other hygiene issues. Urinary catheters be can be uncomfortable, painful, and can cause urinary tract infections. While external catheters improve user comfort, leaks may occur due to size of the user, movement of the external catheter, and/or improper placement of the external catheters.

Thus, users and manufacturers of fluid collection devices continue to seek new and improved devices, systems, and methods to collect urine.

### SUMMARY

Embodiments disclosed herein are fluid collection assemblies including one or more securement elements and methods for collecting fluids with the fluid collection assemblies. In an embodiment, a fluid collection device includes a fluid impermeable barrier, a porous material, and a conduit. The fluid impermeable barrier has an elongated shape with a proximal region and a distal region. The fluid impermeable barrier includes a first impermeable layer and a second impermeable layer opposite to the first impermeable layer and defining an opening. The first impermeable layer and the second impermeable layer at least partially define a chamber therebetween, the chamber including a pocket portion that extends distally from the opening between the first impermeable layer and the second impermeable layer. The opening is sized and positioned to cover substantially all of the vulva of a user. The porous material is disposed in the chamber such that the porous material extends across the opening and is positioned in the pocket portion of the chamber. The porous material is configured to space the first impermeable layer from the second impermeable layer in the pocket portion of the chamber. The conduit extends through the proximal region into the chamber and including an inlet disposed in the pocket portion of the chamber.

In an embodiment, a fluid collection device is disclosed. The fluid collection device includes a fluid impermeable barrier, a porous material, one or more fluid permeable materials, and a conduit. The fluid impermeable barrier has an elongated shape with a proximal region and a distal region. The fluid impermeable barrier includes a first impermeable layer, a second impermeable layer opposite to the first impermeable layer and defining an opening. The first impermeable layer and the second impermeable layer at least partially define a chamber therebetween. The opening is sized and positioned to cover substantially all of the vulva of a user. At least one of the first impermeable layer or second impermeable layer includes a distal portion having the chamber absent therefrom. The porous material is disposed in the chamber such that the porous material extends across the opening and is positioned in the pocket portion of the chamber. The one or more fluid permeable materials cover substantially all of the distal portion of the fluid impermeable barrier. The conduit extends through the proximal region into the chamber and includes an inlet disposed in the chamber.

In an embodiment, a method of manufacturing a fluid collection device is disclosed. The method comprises securing a first fluid impermeable layer to a second fluid impermeable layer having an opening to form a fluid impermeable barrier having an elongated shape with a proximal region, a distal region, and a chamber defined at least partially by the first impermeable layer and the second impermeable layer. The chamber includes a pocket portion that extends distally from the opening between the first impermeable layer and the second impermeable layer. The opening is sized and positioned to cover substantially all of the vulva of a user. The method also includes disposing a porous material in the chamber such that the porous material extends across the opening and is positioned in the pocket portion of the chamber. The porous material spaces the first impermeable layer from the second impermeable layer in the pocket portion of the chamber. The method also includes extending a conduit through the proximal region into the chamber such that an inlet of the conduit is disposed in the pocket portion of the chamber.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is an exploded isometric view of a fluid collection device, according to an embodiment.

FIG. 2A is a front view of a fluid collection device, according to an embodiment.

FIG. 2B is an isometric front view of the fluid collection device of FIG. 2A.

FIG. 2C is a cross-sectional view of the fluid collection device of FIG. 2A.

FIG. 2D is an isometric rear view of the fluid collection device of FIG. 2A in use.

FIG. 3A is an isometric front view of a fluid collection device, according to an embodiment.

FIG. 3B is an isometric rear view of the fluid collection device of FIG. 3A.

FIG. 3C is a cross-sectional view of the fluid collection device of FIG. 3A.

FIG. 4A is an isometric front view of a fluid collection device, according to an embodiment.

FIG. 4B is an isometric rear view of the fluid collection device of FIG. 4A.

FIG. 4C is a cross-sectional view of the fluid collection device of FIG. 4A.

FIG. 5A is an isometric front view of a fluid collection device, according to an embodiment.

FIG. 5B is a front view of the fluid collection device of FIG. 5A.

FIG. 5C is a cross-sectional view of the fluid collection device of FIG. 5A.

FIG. 6A is a front view of a fluid collection device, according to an embodiment.

FIG. 6B is a front view of the fluid collection device of FIG. 6A with a fluid permeable membrane removed.

FIG. 6C is a rear view of the fluid collection device of FIG. 6A with the fluid permeable membrane removed.

FIG. 7A is a front view of a fluid collection device, according to an embodiment.

FIG. 7B is a front view of the fluid collection device of FIG. 7A with a fluid permeable membrane removed.

FIG. 7C is a front view of the fluid collection device of FIG. 7A with the fluid permeable membrane and an absorbent material removed.

FIG. 7D is a cross-sectional view of a portion of the fluid collection device shown in FIG. 7C with the fluid permeable membrane and the absorbent material removed.

FIG. 8 is a block diagram of a system for fluid collection, according to an embodiment.

FIG. 9 is a flow diagram of a method of manufacturing a fluid collection device, according to an embodiment.

### DETAILED DESCRIPTION

Embodiments disclosed herein are pad-shaped external catheters for fluid collection devices, and related systems and methods of collecting fluid. Embodiments of the fluid collection devices disclosed herein are configured to collect fluids from an individual. The fluids collected by the fluid collection devices may include urine. The fluids collected by the fluid collection devices may also include at least one of vaginal discharge, penile discharge, reproductive fluids, blood, sweat, or other bodily fluids. Fluid collection devices such as external catheters improve user comfort over conventional internal catheters.

In at least one, some, or all embodiments described herein, a pad-shaped (e.g., oval or cassini oval) female external catheter (e.g., pad-shaped fluid collection device) combines the wearability and comfort of a female hygiene pad with the suction and extended use benefits of an at least partially negative pressure fluid collection systems, thereby providing more comfort and discretion for the user. Conventional fluid collection devices may become loose (e.g., mispositioned) with too much movement. Women in wheelchairs who are able to navigate daily life and still perform many activities may need a more secure and reliable fluid collection device. Moreover, conventional feminine hygiene pads have limited capacity for urine and require multiple changes per day. Embodiments of fluid collection device described herein solve these issues by having a shape and dimensions that allow the fluid collection devices to better stay in place (and, in some embodiments, by adhering to the underwear of the user), while also allowing for all day use by including a cavity and connection for suction removal of urine from the fluid collection devices.

According to some embodiments, the fluid collection devices described herein include (1) a fluid permeable pad (e.g. fluid permeable body) having one or more wicking materials and (2) a fluid impermeable barrier at least partially defining a slim, contoured cavity on the back side for collecting urine and connecting to a urine collection canister. In some embodiments, the fluid impermeable barrier may be at least partially rigid to such that the fluid impermeable barrier retains its original shape after being manipulated. In some embodiments, the fluid impermeable barrier may include an impermeable film. Example materials of the fluid permeable body and fluid impermeable barrier are provided in greater detail below.

Embodiments of the fluid collection device described herein may be used with females having limited mobility (e.g. wheelchair-bound or walker) and/or at least partially mobile women who may wear the fluid collection device all day in their underwear for comfort and/or protection. In some embodiments, the fluid collection device may include an adhesive on the back of the fluid impermeable barrier configured to hold the fluid collection device in a desired position during use. Embodiments of the fluid collection devices disclosed herein may be used in fluid collection systems. The fluid collection systems may include a fluid collection device, a fluid storage container, and a vacuum source. Fluid (e.g., urine or other bodily fluids) collected in the fluid collection device may be removed from the fluid collection device via a conduit which protrudes into an interior region of the fluid collection device. Fluid collection devices described herein may be shaped and sized to be positioned adjacent to the opening of a female urethra or positioned over a male penis. For example, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra. The fluid collection device may include a fluid permeable body disposed within the fluid impermeable barrier. The conduit may extend into the fluid collection device at a first end region, through one or more of the fluid impermeable barrier and/or the fluid permeable body to a second end region of the fluid collection device. Exemplary fluid collection devices for use with the systems and methods herein are described in more detail below. Fluid, such as urine, may be drained from the fluid collection device using the vacuum source.

In some examples, the fluid collection devices described herein are an examples of a female fluid collection devices configured to receive fluids from a female. The fluid collection device includes a fluid impermeable barrier having a first end region and a second end region. The fluid impermeable barrier at least partially defines a chamber (e.g., interior region), and includes a border or edge defining an opening. The opening is formed in and extends longitudinally through the fluid impermeable barrier, thereby enabling fluids to enter the chamber from outside of the fluid collection device. The opening may be configured to be positioned at least proximate (e.g., adjacent to, interfacing, or contacting) the opening of a female urethra.

With the fluid collection device positioned at least proximate to the opening of the female urethra, urine may enter the interior region of the fluid collection device via the opening. The fluid collection device is configured to receive the fluids into the chamber via the opening. For example, the opening may exhibit an elongated shape that is configured to extend from a first location below the urethral opening (e.g., at or near the anus or the vaginal opening) to a second location above the urethral opening (e.g., at or near the clitoris or the pubic hair). The opening may exhibit an elongated shape since the space between the legs of a female is relatively small when the legs of the female are closed, thereby only permitting the flow of the fluids along a path that corresponds to the elongated shape of the opening. For example, the opening may extend longitudinally along the fluid impermeable barrier. In an embodiment, the fluid impermeable barrier may be configured to be attached to the individual, such as adhesively attached (e.g., with a hydrogel adhesive) to the individual.

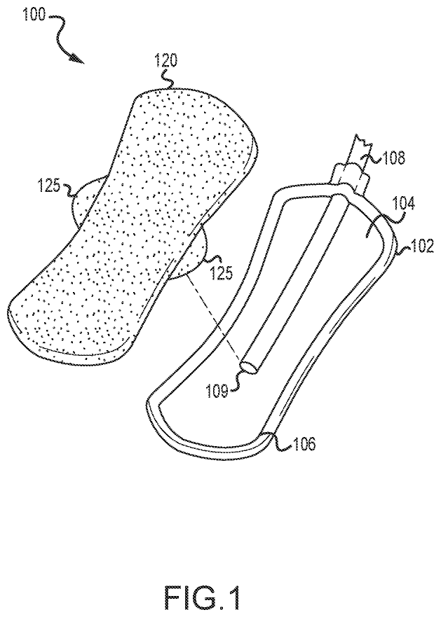

FIG. 1 is an exploded isometric view of a fluid collection device 100, according to an embodiment. The fluid collection device provides discretion and comfort in an improved design that combines the wearability and comfort of a pad with the suction and extended use benefits of at least partially negative pressure urine collection systems. In some embodiments, fluid collection device includes a fluid permeable body 120 and a fluid impermeable barrier 102. The fluid permeable body 120 (e.g. pad) may include a shape and/or configuration of a urinary incontinence pad with the wicking materials. The fluid impermeable barrier 102 may include or define a slim, contoured, cavity (e.g. chamber) on the back side thereof for collecting urine and connecting to the collection canister. The fluid impermeable barrier 102 may be at least somewhat rigid, allowing the fluid impermeable to return to an original shape and configuration after bending. The fluid collection device 100 may be used with women having limited (e.g., wheelchair bound) and/or full mobility. Women may discretely wear the fluid collection device all day in their underwear for comfort and protection.

Conventional negative pressure fluid collection devices may come loose or otherwise mispositioned with too much movement by the user. For women who are in wheelchairs and are able to navigate daily life and still perform many activities, the fluid collection device 100 shown in FIG. 1 provides a more secure and reliable solution. Additionally, with conventional incontinence pads, the incontinence pads have a limited capacity for urine and need to be changed multiple times a day. The fluid collection device 100 shown in FIG. 1 is shaped and configured like a pad, thereby allowing the fluid collection device 100 to securely stay in place by adhering to the underwear. The cavity (e.g., chamber 104) and connection for suction of the fluid collection device allow the fluid collection to be suitable for all day use.

In some embodiments, the fluid collection device 100 includes a fluid impermeable barrier 102 having a first end region and a second end region. The fluid impermeable barrier 102 at least partially defines a chamber 104 (e.g., interior region) and includes an inward border or edge defining an opening 106. The fluid impermeable barrier 102 may be substantially planar or flat on the back side of the device 100 opposite to the opening 100. In other embodiments, the fluid impermeable barrier 102 may include other shapes. The opening 106 is formed in and extends longitudinally through the fluid impermeable barrier 102, thereby enabling fluids to enter the chamber 104 from outside of the fluid collection device 100. The opening 106 may be configured to be positioned at least proximate (e.g., adjacent to, interfacing, or contacting) the opening of a female urethra or over a penis of a male. The opening 106 may extend across substantially all of a front side of the fluid collection device 100.

With the fluid collection device 100 positioned at least proximate to the opening of the female urethra or over a penis, urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., the perineum, at or near the anus, or below the vaginal opening) to a second location above the urethral opening (e.g., at or near the pubic bone). In some embodiments, the opening 106 is sized such that the fluid permeable body 120 covers substantially all of the vulva of a female user. In an embodiment, the fluid impermeable barrier 102 may be configured to be attached to the individual, such as adhesively attached (e.g., with a hydrogel adhesive) to the individual.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, a closed cell foam, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick or otherwise draw any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 can be configured to wick and/or allow transport of fluid away from the opening 106 towards a reservoir and/or an inlet 109 of the conduit 108. The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” or other physical properties may exclude absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 120 (e.g., absorbency), such as less than about 30 wt % of the dry weight of the fluid permeable body 120, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 120 may include at least one absorbent or adsorbent material.

The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir or sump in the chamber 104 and/or the inlet 109 of the conduit 108. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating.

The fluid permeable membrane may also wick the fluid generally towards an interior of the chamber 104. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous spun nylon fiber structure and a fluid permeable wicking membrane including gauze at least partially enclosing the spun nylon fiber structure. For example, the fluid permeable body 120 may include a gauze or other wicking fabric positioned to contact the skin of the user through the opening 106. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

In some embodiments, the fluid permeable body 120 may include any of the fluid permeable, absorbent, or porous materials described herein. For example, in some embodiments, the fluid permeable body 120 includes one more layers of the porous material 710 and a fluid permeable membrane covering the one or more layers of the porous material 710.

At least one of the fluid permeable body 120 or the fluid impermeable barrier 102 may include wings 125 configured to help secure the fluid collection device 100 in a position to collect fluid. For example, the wings 125 may include an adhesive configured to secure to at least one of the inner thighs of the user or garments of the wearer covering the fluid collection device 100. Accordingly, the wings 125 may be configured to fold to adhere to at least one of the inner thighs of the user or garments of the wearer covering the fluid collection device 100.

The fluid collection device 100 also includes the conduit 108 that is at least partially disposed in the chamber 104. The conduit 108 (e.g., a tube) includes an inlet 109 at a second end region of the fluid impermeable barrier 102 and may include an outlet at a first end region of the fluid impermeable barrier 102 positioned downstream from the inlet 109. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir with the fluid storage container or the portable vacuum source.

In some embodiments, the fluid permeable body 120 defines a bore extending through the fluid permeable body 120 from a first body end of the fluid permeable body 120 to a second body end of the fluid permeable body 120 distal to the first body end. In other embodiments, the bore extends only partially into the fluid permeable body 120 from the first body end of the fluid permeable body 120.

In some embodiments, the conduit 108 is at least partially disposed in the chamber 104 and interfaces at least a portion of the bore of the fluid permeable body 120. For example, the conduit 108 may extend into the fluid impermeable barrier 102 from the first end region (e.g., proximate to the outlet) and may extend through the bore to the second end region (e.g., opposite the first end region) to a point proximate to the reservoir such that the inlet 109 is in fluid communication with the reservoir. For example, in the illustrated embodiment, the inlet 109 extends past the second body end and is positioned in the reservoir. However, in other embodiments, the inlet 109 may be positioned flush with or behind the second body end of the fluid permeable body 120 that partially defines the reservoir. In some embodiments, the second body end extends to the second end region to substantially fill the chamber 104 and cover the inlet 109. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir therein. The reservoir may be an unoccupied portion of the chamber 104 and is void of other material. In some embodiments, the reservoir is defined at least partially by the fluid permeable body 120 and the fluid impermeable barrier 102. For example, in an embodiment, the reservoir may be located at the portion of the chamber 104 that is closest to the inlet 109 (e.g., the second end region). However, the reservoir may be located at different locations in the chamber 104. For example, the reservoir may be located at the end of the chamber 104 that is closest to the outlet. In these and other embodiments, the conduit 108 may extend through the first end region of the fluid impermeable barrier 102 and to the reservoir without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 109 (e.g., second end region) and a second reservoir that is located at the portion of the of the chamber 104 that is closest to the outlet (e.g., first end region). In another example, the fluid permeable body 120 is spaced from at least a portion of the conduit 108 and the reservoir may be the space between the fluid permeable body 120 and the conduit 108. In some embodiments, the fluid permeable body 120 fills or occupies substantially all of the chamber 104, including filling or occupying substantially all of the reservoir is between the inlet 109 and the second end region of the fluid impermeable barrier 102. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore. In another example, the fluid impermeable barrier 102 may define an aperture sized to receive the conduit 108 (e.g., at least one tube). The at least one conduit 108 may be disposed in the chamber 104 via the aperture. The aperture may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir, as shown in FIG. 1. In some embodiments, the conduit 108 may extend through the fluid permeable body 120 and terminate at or before the second body end of the fluid permeable body 120 such that the conduit 108 does not extend into the reservoir (or the reservoir is absent of the conduit 108). For example, an end (e.g., the inlet 109) of the conduit 108 may be generally flush or coplanar with the second body end of the fluid permeable body 120. In other embodiments, the end of the conduit 108 may be recessed from the second body end of the fluid permeable body 120. The end (e.g., the inlet 109) of the conduit 108 also may be selectively moveable between partially extending into the reservoir and recessed from or flush with the second body end 123 of the fluid permeable body.

When secured to the fluid collection device 100, the conduit 108 is configured to provide fluid communication with and at least partially extend between one or more of a fluid storage containers and a portable vacuum source. For example, the conduit 108 may be configured to be fluidly coupled to and at least partially extend between one or more of the fluid storage containers and the portable vacuum source. In an embodiment, the conduit 108 is configured to be directly connected to the portable vacuum source. In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container or the portable vacuum source. In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 109 and the outlet are configured to provide fluid communication (e.g., directly or indirectly) between the portable vacuum source (not shown) and the chamber 104 (e.g., the reservoir). For example, the inlet 109 and the outlet of the conduit 108 may be configured to directly or indirectly fluidly couple the portable vacuum source to the reservoir. In an embodiment, the inlet 109 and/or the outlet may form a male connector. In another example, the inlet 109 and/or the outlet may form a female connector. In an embodiment, the inlet 109 and/or the outlet may include ribs that are configured to facilitate secure couplings. In an embodiment, the inlet 109 and/or the outlet may form a tapered shape. In an embodiment, the inlet 109 and/or the outlet may include a rigid or flexible material.

Locating the inlet 109 at or near a gravimetrically low point of the chamber 104 enables the conduit to receive more of the fluids than if inlet 109 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir positioned at the first end region, the second end region, or other intermediary positions within the chamber 104) may be drawn into the inlet 109 and out of the fluid collection device 100 via the conduit 108.

In an embodiment, the conduit 108 is configured to be at least insertable into the chamber 104. In such an embodiment, the conduit 108 may include one or more markers on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 109 that is configured to be disposed in or adjacent to the reservoir. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet) and may extend to the second end region (e.g., opposite the first end region) to a point proximate to the reservoir such that the inlet 109 is in fluid communication with the reservoir. In some embodiments (not shown), the conduit 108 may enter the second end region and the inlet 109 may be disposed in the second end region (e.g., in the reservoir). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer. In some embodiments, an odor absorber such as carbon or sodium bicarbonate may be including with the fluid collection device.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device. In some embodiments, a sensor to detect stool also may be included in the fluid collection device.

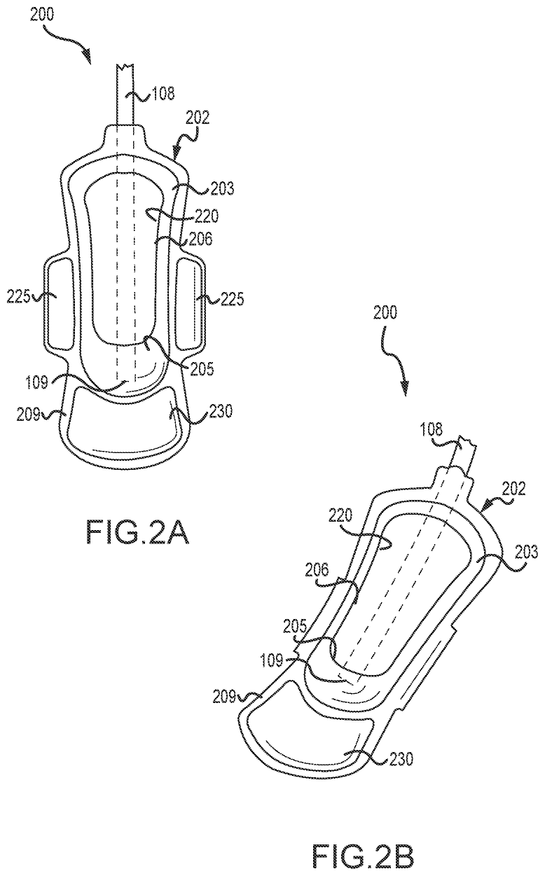

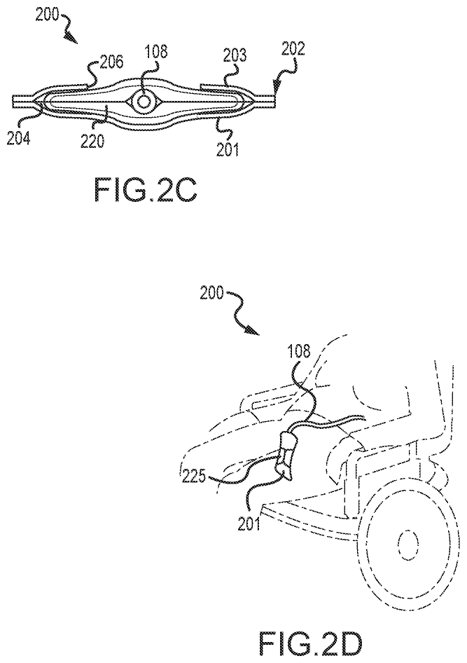

FIGS. 2A-2D show various views and use of a fluid collection device 200, according to an embodiment. Specifically, FIG. 2A is a front view of a fluid collection device 200, FIG. 2B is an isometric front view of the fluid collection device 200 of FIG. 2A, FIG. 2C is a cross-sectional view of the fluid collection device 200 of FIG. 2A, and FIG. 2D is an isometric rear view of the fluid collection device 200 of FIG. 2A in use, according to an embodiment. The fluid collection device 200 may be contoured and compliant, and include folding wings 225.

The fluid collection device 200 may include a fluid impermeable barrier 202 extending across a back of the fluid collection device 200 and a portion of the front of the fluid collection device 200, thereby at least partially defining an opening 206 on the front of the fluid collection device 200. The fluid impermeable barrier 202 may include heat-sealed on one or more edges to prevent leaks around a perimeter of the fluid collection device 200. For example, the fluid impermeable barrier 202 may include a first layer 201 forming a back of the fluid collection device 200 and a second layer 203 forming a portion of the front of the fluid collection device 200. The second layer 203 may at least partially (e.g., entirely) define the opening 206. The first layer 201 and the second layer 203 may each at least partially define the chamber 204, and the first layer 201 and the second layer 203 may be heat-sealed on one or more edges to prevent leaks from the chamber 204.

In some embodiments, the chamber 204 includes a pocket portion 205 that extends distally from the opening 206 between the first impermeable layer 201 and the second impermeable layer 203. The pocket portion 205 includes the region of the chamber 204 from the distal end of the opening 206 until the chamber 204 terminates distal to the distal end of the opening 206. Accordingly, the pocket portion 205 of the chamber 204 may be defined or bordered by the first impermeably layer 201, the second impermeable layer 203, and a terminating end of the chamber 204 where the first impermeable layer 201 and the second impermeable layer 203 are heat-sealed, adhered to one another, or otherwise secured together. In some embodiments, the pocket portion 205 extends distally from the distal end of the opening 206 to a terminating end of the chamber 204 at a length of at least about 2.5 cm, at least about 5 cm, at least about 7.5 cm, about 2 cm to about 5 cm, about 2.5 cm to about 4.5 cm, about 3.5 cm to about 5.5 cm, about 4.5 cm to about 6.5 cm, about 5.5 cm to about 7.5 cm, about 2.5 cm to about 3.5 cm, about 3.5 cm to about 4.5 cm, about 4.5 cm to about 5.5 cm, about 5.5 cm to about 6.5 cm, or about 6.5 cm to about 7.5 cm.

The first layer 201 and the second layer 203 of the fluid impermeable barrier 202 may include any of the fluid impermeable materials described above. In some embodiments, the first layer 201 and the second layer 203 each include a plastic impermeable layer, such as a fluid impermeable polyurethane film.

A fluid permeable body 220 is positioned within a chamber 204 or cavity defined by the fluid impermeable barrier 202. In some embodiments, the fluid permeable body fills substantially all of the chamber 204, including substantially all of the pocket portion 205 of the chamber 204. The fluid permeable body 220 may include any of the fluid permeable materials described herein. In many embodiments, the fluid permeable body 220 includes a fluid permeable support including a porous spun nylon fiber structure and a fluid permeable wicking membrane including gauze at least partially enclosing or covering the spun nylon fiber structure. For example, the fluid permeable body 220 may include a gauze or other wicking fabric positioned to contact the skin of the user through the opening 206. In some embodiments, the gauze or other wicking fabric is wrapped around or disposed on a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

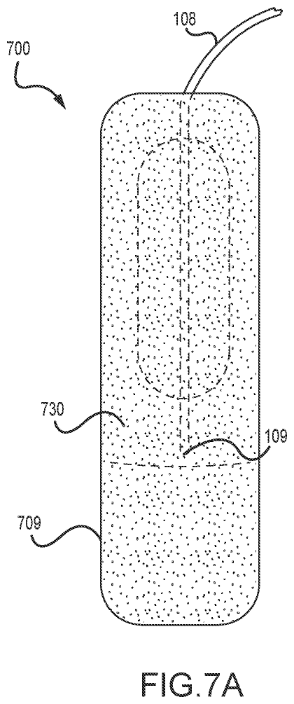

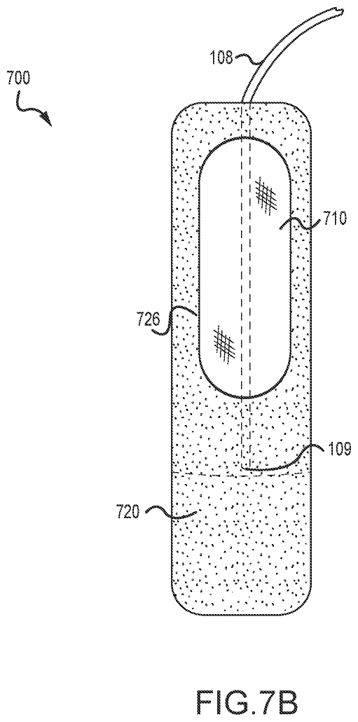

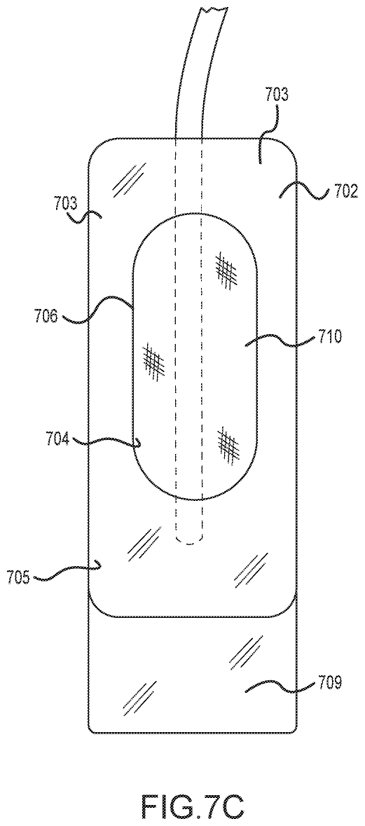

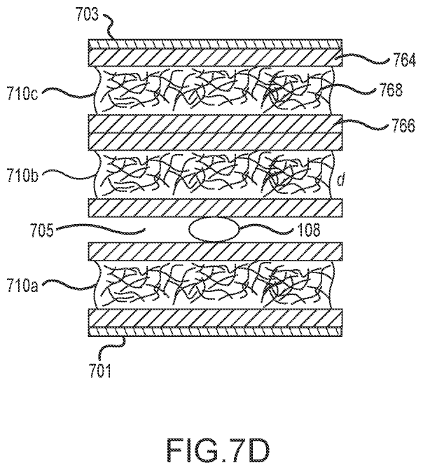

In some embodiments, the fluid permeable body 220 includes one or more sheets of porous material. FIG. 7D, for example, shows a cross-sectional schematic of three sheets of a porous material 710 that may be positioned in the chamber 204 of the fluid collection device 200. The sheets of the porous material 710 may include a spacer fabric that imparts a three-dimensional property to the chamber 204 to space the first layer 201 from the second layer 203 in the chamber 204. In some embodiments, the porous material 710 includes a first layer 764 and a second layer 766. The first and second layers 764, 766 may be a woven material. The porous material 710 also includes a plurality of fibers 768 forming a layer (e.g., intermediary layer) between the first layer 764 and the second layer 766. Each of the first layer 764, the second layer 766, and the plurality of fibers 768 define a plurality of pores, thereby allowing transport of the bodily fluids and air circulation through the porous material 710. The pores defined by the plurality of fibers 768 may be at least one of larger or more numerous, thereby decreasing the likelihood that dried bodily fluids clog the porous material 710. The presence of the plurality of fibers 768 also cause the porous material 710 feel soft against the skin of the user and provides a cushioning effect to the skin. The plurality of fibers 768 may also prevent the vacuum force from collapsing the porous material 710 in the chamber 204.

In an embodiment, the plurality of fibers 768 may cause the first layer 764 to be spaced from the second layer 766 by a distance d. The distance d may be selected based on the number of fibers that form the plurality of fibers 768 and the density at which the plurality of fibers 768 are packed together. For example, the distance d may be selected to be about 0.25 mm or more, about 0.5 mm or more, about 0.75 mm or more, about 1 mm or more, about 1.5 mm or more, about 2 mm or more, about 3 mm or more, about 4 mm or more, about 5 mm or more, about 6 mm or more, about 8 mm or more, about 10 mm or more, about 12.5 mm or more, about 15 mm or more, or in ranges of about 0.25 mm to about 0.75 mm, about 0.5 mm to about 1 mm, about 0.75 mm to about 1.5 mm, about 1 mm to about 2 mm, about 1.5 mm to about 3 mm, about 2 mm to about 4 mm, about 3 mm to about 5 mm, about 4 mm to about 6 mm, about 5 mm to about 8 mm, about 6 mm to about 10 mm, about 8 mm to about 12.5 mm, or about 10 mm to about 15 mm. The thickness of the distance d may be selected to adjust the absorbency of the porous material 710. For example, increasing the thickness may increase the volume of the plurality of fibers 768 and/or the porosity defined by the plurality of fibers 768 which increases the amount of bodily fluids that may be received by and at least partially stored in the porous material 710.

The first layer 764, the second layer 766, and the plurality of fibers 768 may be formed from any suitable material, such as a hydrophobic material, a hydrophilic material, polyester, cotton, or any other porous material disclosed herein. In an embodiment, one or more of the first layer 764, the second layer 766, or the plurality of fibers 768 are formed from a hydrophobic material that inhibits the porous material 710 from storing the bodily fluids therein which may facilitate removal of the bodily fluids from the chamber 204. In an embodiment, one or more of the first layer 764, and second layer 766, or the plurality of fibers 768 are formed from a hydrophilic material which allows the porous material 710 to temporarily store the bodily fluids therein thereby limiting the quantity of bodily fluids that pool around the skin of the individual. In an embodiment, two or more of the first layer 764, the second layer 766, or the plurality of fibers 768 are formed from different materials. In such an embodiment, the first layer 764 may define the region of the fluid permeable body 220 that extends across the opening 206 or is otherwise closer to the skin receiving area than the second layer 766. The first layer 764 may be formed from a hydrophobic material while the plurality of fibers 768 are formed from a hydrophilic material. Such a configuration may cause the bodily fluids to be pulled through the first layer 764 and temporarily stored in the plurality of fibers 768. However, the first layer 764 may remain substantially dry due to the hydrophobicity thereof which allows the porous material 710 to feel dry to the skin. In an embodiment, at least one of the first layer 764 or the second layer 766 is formed from a fabric (e.g., fabric gauze) and the plurality of fibers 768 is formed from nylon fibers (e.g., spun nylon fibers). In some embodiments, a plurality of sheets of the porous material 710 are disposed in the chamber 204 to form the fluid permeable body 220.

Returning to FIGS. 2A-2D, the conduit 108 may extend through a proximal region of the fluid impermeable barrier 202 and the inlet 109 of the conduit 108 may be disposed in, for example, the pocket portion 205 of the chamber 204. Accordingly, the inlet 109 may be disposed between a distal end of the opening 206 and a distal end of the chamber 204. In some embodiments, the inlet 109 is disposed between the distal end of the opening 206 and a proximal end of the opening 206. The conduit 108 may extend between portions of the fluid permeable body 220 such that the conduit 108 is generally surrounded by the fluid permeable body 220. In some embodiments, the conduit 108 extends between the fluid permeable body 220 and the first layer 201 of the fluid impermeable barrier 202.

In some embodiments, the fluid collection device 200 includes one or more wings 225. The wings 225 of the fluid collection device 200 may include an adhesive to secure the fluid collection device 200 to the clothes or body of the user (shown in FIG. 2D).

In some embodiments, at least one of the first impermeable layer 201 or second impermeable layer 203 of the fluid impermeable barrier 202 includes a distal portion 209 that extends distally from the pocket portion 205 with the chamber 204 being absent from the distal portion 209. An additional fluid permeable body 230 may be disposed on the distal portion 209 of the fluid impermeable barrier. For example, at least one of an absorbent material and/or a wicking material may be disposed on the distal portion 209 to at least partially cover the distal portion 209. In some embodiments, at least the absorbent material is disposed on the distal portion 209 to at least partially cover the distal portion 209. The absorbent material may include a non-woven material, such as a non-woven polyester material, an open cell foam (e.g., polyurethane, diisocyanate, hydrophilic-treated polyethylene), cellulose fibers, non-woven or woven fibers, and/or gel sorbents (e.g., crosslinked sodium polyacrylate polymer beads/granules). In some embodiments, the absorbent material may include a foam or woven material. In some embodiments, both the absorbent material and also the wicking material may be disposed on the distal portion 209 to at least partially cover the distal portion 209. The wicking material may include any of the wicking materials or fluid permeable membranes described herein. In an embodiment, the wicking material includes a polyimide material.

In use, the opening 206 may be positioned over at least the urethra of the user (and, in some embodiments, over the vulva of the user) such that fluid voided by the user is directed into through the opening 206 into the fluid permeable body 220. Fluid may flow downward in the fluid permeable body to the pocket portion 205 of the chamber 204 where the inlet 109 of the conduit 108 is positioned. Application of an at least partially negative pressure to the conduit 108 draws the fluid in the pocket portion 205 into the inlet 109 of the conduit 108 for removal from the fluid collection device 200. The distal portion 209 is positioned such that any overflow fluid or misdirected fluid is absorbed by the additional fluid permeable body 230.

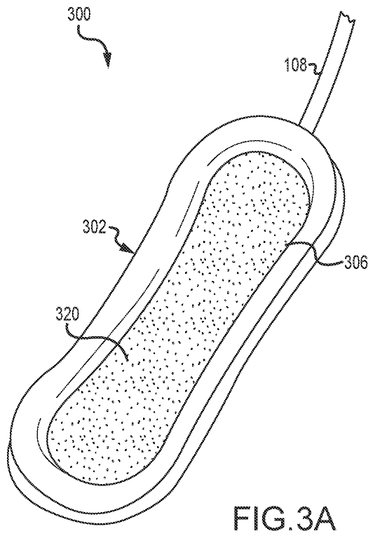

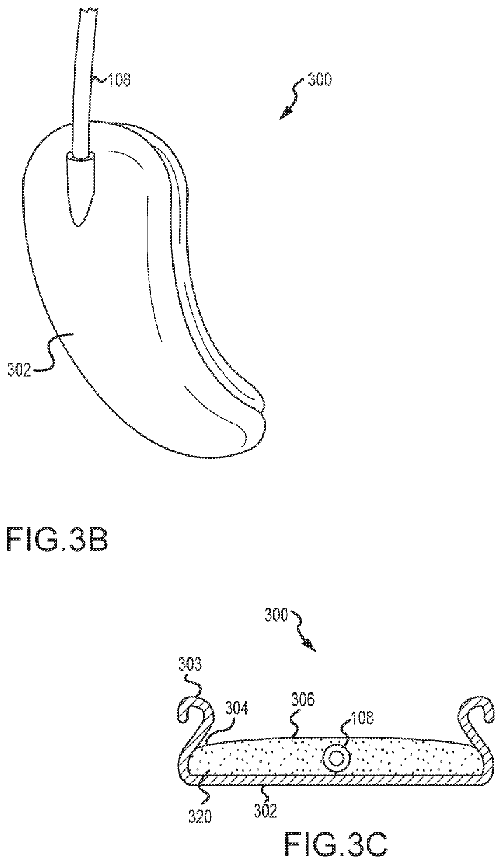

FIGS. 3A-3C shows various views and use of a fluid collection device 300, according to an embodiment. Specifically, FIG. 3A is an isometric front view of the fluid collection device 300, FIG. 3B is an isometric rear view of the fluid collection device 300 of FIG. 3A, and FIG. 3C is a cross-sectional view of the fluid collection device 300 of FIG. 3A. The fluid collection device includes a fluid impermeable barrier 302 defining a chamber 304 or cavity, and a fluid permeable body 320 disposed at least partially within the chamber 304. The fluid impermeable barrier 302 may include any of the fluid impermeable materials described herein. In some embodiments, the fluid impermeable barrier 302 includes a thermoplastic elastomer material having a soft, deformable perimeter flange 303. The surface of the fluid permeable body 320 may be recessed relative to the deformable perimeter flange 303. As shown in FIG. 3C, the perimeter flange 303 may curl outward from the chamber 304. In some embodiments, the side walls of the fluid impermeable barrier 302 may orient inward from the back of the fluid impermeable barrier 202 towards the interior of the chamber 304 before curling outward from the chamber 304 and turning towards the back of the fluid impermeable barrier 302. This configuration may provide a built-in cushion for comfort against the skin of the user.

The fluid permeable body 320 may include any of the fluid permeable materials described herein, such as any of the materials (including combinations) described in relation to the fluid permeable support, the fluid permeable membrane, the wicking material, and/or absorbent material. The fluid collection device 300 may include a shape and configuration that allows the fluid collection device to be held in a desired position at least proximate to a urethra of a user, between the clothes and anatomy of the user. The conduit 108 may extend through a proximal region of the fluid collection device 300 and the inlet 109 of the conduit 109 may be disposed in a distal region of the chamber 304. As shown in FIG. 3C, the conduit 108 may extend through a bore in the fluid permeable body 320.

In use, the opening 306 may be positioned over at least the urethra of the user (and, in some embodiments, over the vulva of the user) such that fluid voided by the user is directed into through the opening 306 into the fluid permeable body 320. Fluid may flow downward in the fluid permeable body 320 to a distal region of the fluid collection device 300 where the inlet 109 of the conduit 108 is positioned. The recess of the fluid permeable body 320 relative to the perimeter flange 303 inhibits fluid from leaking from the fluid collection device 300. Application of an at least partially negative pressure to the conduit 108 draws the fluid into the inlet 109 of the conduit 108 for removal from the fluid collection device 300.

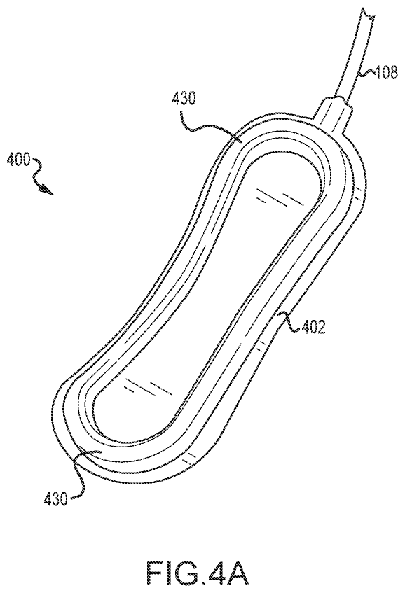

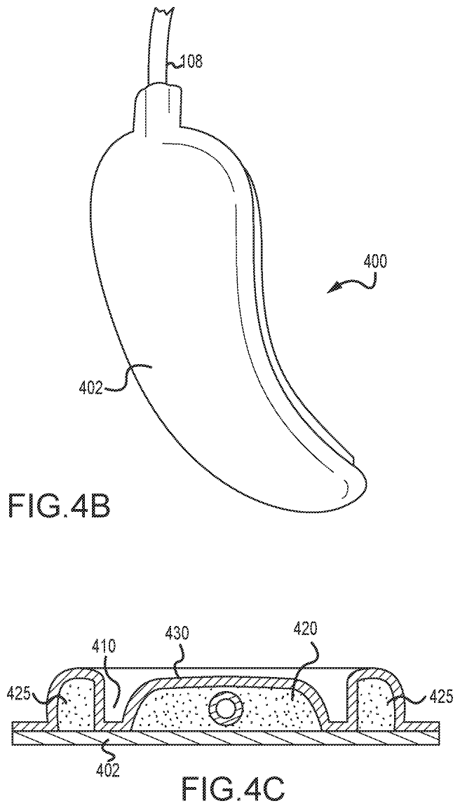

FIGS. 4A-4C show various views and uses of a fluid collection device 400, according to an embodiment. Specifically, FIG. 4A is an isometric front view of the fluid collection device 400, FIG. 4B is an isometric rear view of the fluid collection device 400 of FIG. 4A, and FIG. 4C is a cross-sectional view of the fluid collection device 400 of FIG. 4A. The fluid collection device 400 may be contoured and compliant, having a soft, compliant foam perimeter 425 (shown under the fluid permeable membrane 430 in FIG. 4A). The fluid collection device 400 also may include a fluid impermeable backing 402 (e.g., fluid impermeable layer) and a fluid permeable body 420 (e.g., fluid permeable layer, shown under the fluid permeable membrane 430 in FIG. 4A). At least one (e.g., both) of the fluid permeable body 420 and the foam perimeter 425 may be heat-sealed to the fluid impermeable backing 402. In some embodiments, a wicking layer or fluid permeable membrane 430 may cover the fluid permeable body 420 and/or the foam perimeter 425. The fluid permeable membrane 430 may be heat-sealed, adhered, or otherwise secured to the fluid impermeable backing 402.

The material of the fluid impermeable backing 402 may include any fluid impermeable material described herein. In some embodiments, the fluid impermeable backing 402 includes a fluid impermeable polyurethane film. The material of the fluid permeable body 420 may include any fluid permeable material described herein. In some embodiments, the fluid permeable body includes a fiber material, such as spun nylon fibers material. The material of the fluid permeable membrane 430 may include any wicking material or fluid permeable membrane described herein. In some embodiments, the fluid permeable membrane 430 includes gauze or other wicking fabric. The material of the foam perimeter 425 includes a fluid impermeable foam or non-absorbent foam material, such as a skin foam or a closed cell foam.

The foam perimeter 425 and the fluid permeable body 420 may protrude or extend from the fluid impermeable backing 402. In some embodiments, the foam perimeter 425 forms a ring around the fluid permeable body 420, with an annular recess 410 being formed between the foam perimeter 425 and the fluid permeable body 420. The height of the foam perimeter 425 from the fluid impermeable backing 402 may be greater than the height of the fluid permeable body 420 from the fluid impermeable backing 402. In use, the foam perimeter 425 may prevent leaks of discharged fluid from the fluid collection device 400, and/or may direct discharged fluids towards the fluid permeable body 420 for removal from the fluid collection device 400.

A conduit 108 (e.g., tube) may extend into the fluid permeable body 420. The fluid collection device 400 may include a shaped and configured that allows the fluid collection device to be secured in a desired positioned at least proximate to the urethra of the user between the clothes and anatomy of the user. In some embodiments, straps may be used to secure the fluid collection device in place.

In use, the fluid collection device 400 is positioned such that the fluid permeable body 420 is over at least the urethra of the user (and, in some embodiments, over the vulva of the user) and fluid voided by the user is directed into through the fluid permeable membrane 430 into the fluid permeable body 420. Fluid may flow downward in the fluid permeable body 420 to a distal region of the fluid collection device 400 where the inlet 109 of the conduit 108 is positioned. The annular recess 410 between the fluid permeable body 420 relative and the foam perimeter 425 inhibits fluid from leaking from the fluid collection device 400. Application of an at least partially negative pressure to the conduit 108 draws the fluid into the inlet 109 of the conduit 108 for removal from the fluid collection device 400.

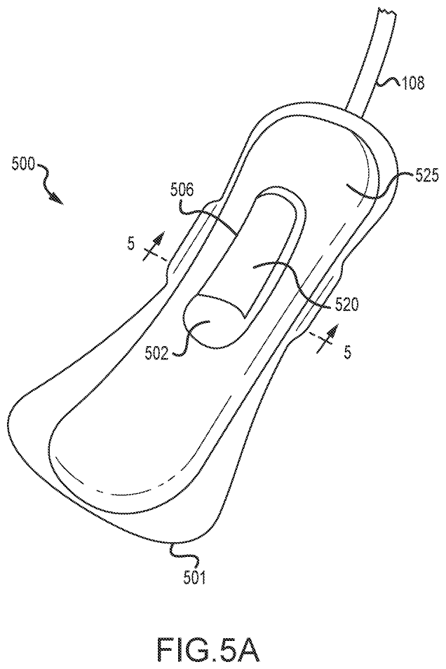

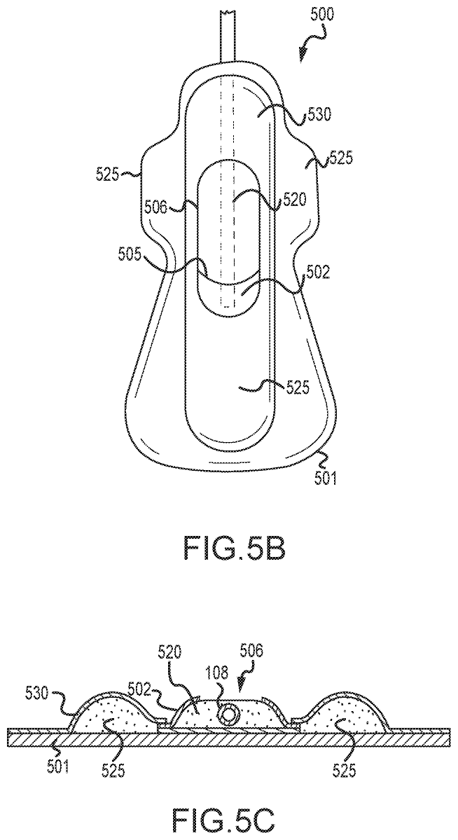

FIGS. 5A-5C show various views of a fluid collection device 500, according to an embodiment. Specifically, FIG. 5A is an isometric front view of the fluid collection device 500, FIG. 5B is a front view of the fluid collection device 500 of FIG. 5A, and FIG. 5C is a cross-sectional view of the fluid collection device 500 of FIG. 5A. The fluid collection device 500 may include a fluid impermeable barrier 502 surrounded on the perimeter of the fluid impermeable barrier 502 at least partially (e.g., entirely) by one or more padded portions 525 (shown under the fluid permeable membrane 530 in FIGS. 5A-5B). The fluid impermeable barrier 502 may at least partially defining an opening 506 positioned on the fluid collection device 500 to receive urine or other fluid discharged by a user. A fluid permeable body 520 may be disposed in the chamber or cavity defined by the fluid impermeable barrier 502, and a conduit 108 (e.g. tube) may extend at least partially into the chamber or cavity. In some embodiments, the fluid impermeable barrier 502 forms a pocket portion 505 of the chamber that extends distally from the opening 506. The pocket portion 505 may include any aspect of the pocket portion 205 described above. The inlet of the conduit 108 may be disposed in the pocket portion 505. The fluid impermeable barrier 502, the fluid permeable body 520, and/or the padded portions 525 may be secured or securable to a fluid impermeable backing 501. A fluid permeable membrane 530 may cover one or more (e.g., all) of the padded portions 525, the fluid permeable body, and/or the fluid impermeable barrier 502 opposite of the fluid impermeable backing 501. Fluid discharged into the chamber of cavity may be suctioned from the chamber or cavity through the conduit. In some embodiments, a fluid permeable body 520 is absent from the chamber or cavity of the fluid impermeable barrier 502.

The fluid impermeable barrier 502 may include any fluid impermeable material described herein. In some embodiments, the fluid impermeable barrier 502 includes silicone and/or TPE, and may be semi-rigid. In some embodiments, the fluid impermeable barrier 502 includes a fluid impermeable polyurethane film. The fluid impermeable backing 501 may include any of the fluid impermeable materials described herein. In some embodiments, the fluid impermeable backing 501 includes a fluid impermeable polyurethane film. The material of the fluid permeable body 520 may include any fluid permeable or porous material described herein. In some embodiments, the fluid permeable body 520 includes a fiber material, such as spun nylon fibers material. In some embodiments, the fluid permeable body 520 include a porous material, such as the spacer fabric of the porous material 710 described above. The material of the fluid permeable membrane 530 may include any wicking material and/or fluid permeable membrane described herein. In some embodiments, the fluid permeable membrane 530 includes gauze or other wicking fabric. The material of the padded portions 525 may include an absorbent or foam material, such as a non-woven polyester material, an open cell foam (e.g., polyurethane, diisocyanate, hydrophilic-treated polyethylene), cellulose fibers, non-woven or woven fibers, and/or gel sorbents (e.g., crosslinked sodium polyacrylate polymer beads/granules). One or more regions of the padded portion 525 may include an adhesive configured to secure to the user.

In use, the opening 506 may be positioned over at least the urethra of the user (and, in some embodiments, over the vulva of the user) such that fluid voided by the user is directed into through the opening 506 into the fluid permeable body 520. Fluid may flow downward in the fluid permeable body to the pocket portion 505 of the chamber 504 where the inlet 109 of the conduit 108 is positioned. Application of an at least partially negative pressure to the conduit 108 draws the fluid in the pocket portion 505 into the inlet 109 of the conduit 108 for removal from the fluid collection device 500. The one or more padded portions 525 are positioned such that any overflow fluid or misdirected fluid is absorbed by the one or more padded portions 525.

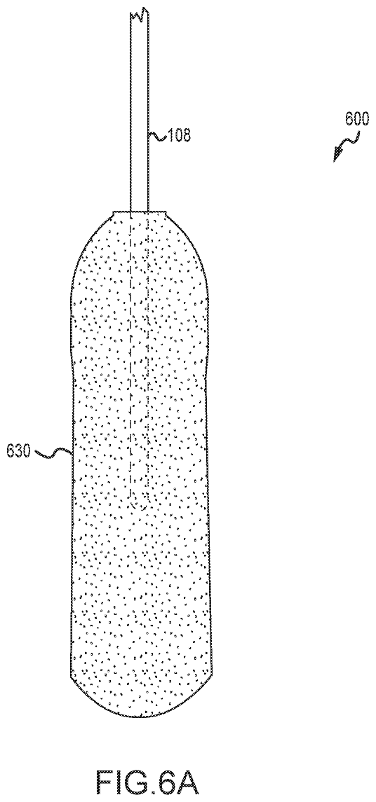

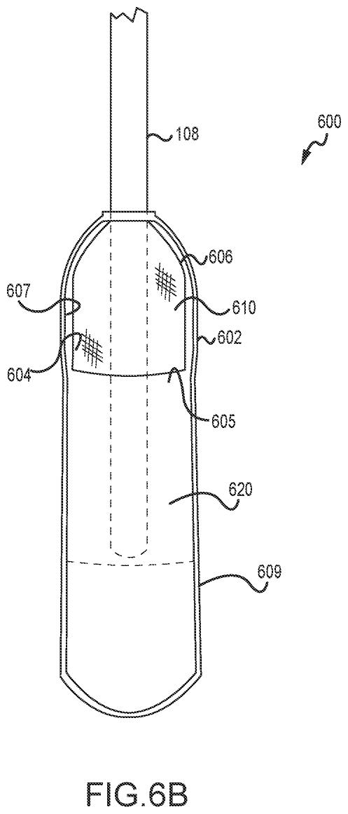

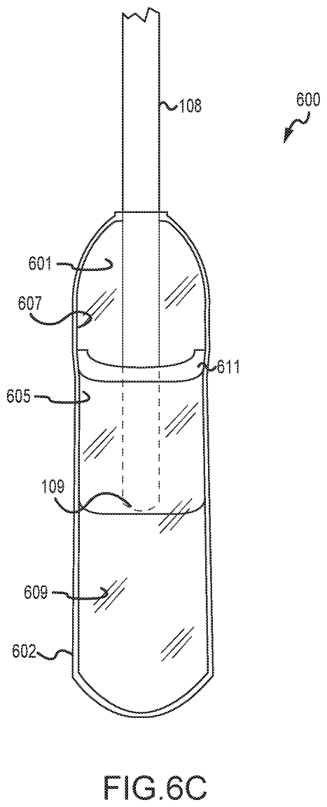

FIGS. 6A-6C show various views of a fluid collection device 600, according to an embodiment. Specifically, FIG. 6A is a front view of the fluid collection device 600, FIG. 6B is a front view of the fluid collection device of FIG. 6A with a fluid permeable membrane 630 removed, and FIG. 6C is a rear view of the fluid collection device 600 of FIG. 6A with the fluid permeable membrane 600 removed. In some embodiments, the fluid collection device 600 includes a fluid impermeable barrier 602, a fluid permeable body 610, and a conduit 108. The fluid collection device 600 also may include one or more additional fluid permeable materials, such as an absorbent material 620 and a fluid permeable membrane 630.

The fluid impermeable barrier 602 may have an elongated shape with a proximal region (near the conduit 108 in FIG. 6A) and a distal region (opposite to the conduit 108 in FIG. 6A). The fluid impermeable barrier 602 may include a first impermeable layer 601 (shown in FIG. 6C), a second impermeable layer (not visible in FIGS. 6A-6C) opposite to the first impermeable layer 601 and defining an opening 606, and a chamber 604 defined at least partially by the first impermeable layer 601 and the second impermeable layer. The chamber 604 may include a pocket portion 605 that extends distally from the opening 606 between the first impermeable layer 601 and the second impermeable layer. The pocket portion 605 includes the region of the chamber 604 from the distal end of the opening 606 until the chamber 604 terminates distal to the distal end of the opening 606. Accordingly, the pocket portion 605 of the chamber 604 may be defined or bordered by the first impermeably layer 601, the second impermeable layer, and a terminating end of the chamber 604 where the first impermeable layer 601 and the second impermeable layer are heat-sealed, adhered to one another, or otherwise secured together. In some embodiments, the pocket portion 605 extends distally from the distal end of the opening 606 to a terminating end of the chamber 604 at a length of at least about 2.5 cm, at least about 5 cm, at least about 7.5 cm, about 2 cm to about 5 cm, about 2.5 cm to about 4.5 cm, about 3.5 cm to about 5.5 cm, about 4.5 cm to about 6.5 cm, about 5.5 cm to about 7.5 cm, about 2.5 cm to about 3.5 cm, about 3.5 cm to about 4.5 cm, about 4.5 cm to about 5.5 cm, about 5.5 cm to about 6.5 cm, or about 6.5 cm to about 7.5 cm.

The opening 606 may be sized and positioned to cover at least the urethra of the user, and may be sized and positioned to cover the vulva of a user. The first layer 601 and the second layer of the fluid impermeable barrier 602 may include any fluid impermeable material described herein. In some embodiments, the first layer 601 and the second layer of the fluid impermeable barrier 602 may include a fluid impermeable polyurethane film.

The fluid permeable body 610 is disposed in the chamber such that the fluid permeable body 610 extends across the opening 606 and is positioned in the pocket portion 605 of the chamber 604. The fluid permeable body 610 may be configured to space the first impermeable layer 601 from the second impermeable layer in the pocket portion 605 of the chamber 604. The fluid permeable body 610 may include any of the fluid permeable materials and/or porous materials described herein. In some embodiments, the fluid permeable body 610 includes a porous spun nylon fiber structure that spaces or separates the first fluid impermeable layer 601 from the second fluid impermeable layer to form the chamber 604 (including the pocket portion 605).

In some embodiments, the fluid permeable body 610 may be absent from the chamber 604, and the fluid collection device 600 may include a peripheral spacer 607 that extends around a perimeter of the chamber 604. The peripheral spacer 607 also may be present in embodiments including a fluid permeable body 610 disposed in the chamber 604. The peripheral spacer 607 may be configured to space the first layer 601 and the second layer of the fluid impermeable barrier 602 from one another to form an open chamber generally void of material. In these and other embodiments, a fluid permeable membrane or other porous material described herein may extend across the opening 606. In some embodiments, the fluid collection device 600 also may include a support member 611 extending laterally across the fluid collection device 600 proximate to the opening 606 and the pocket portion 605. The support member 611 may be positioned to keep the second layer of the fluid impermeable barrier 602 spaced from the first layer 601 at the border of the opening 606 and the pocket portion 605. The support member 611 may be positioned between the second layer of the impermeable barrier 602 and the peripheral spacer 607, or the support member 611 itself may extend laterally across the chamber 604 and also into the chamber 604 to keep the second layer of the fluid impermeable barrier 602 spaced from the first layer 601 at the border of the opening 606 and the pocket portion 605.

The conduit 108 may extend through the proximal region of the fluid impermeable barrier 602 into the chamber 604. In some embodiments, the inlet 109 of the conduit 108 is disposed in the pocket portion 605 of the chamber 604. In some embodiments, the conduit 108 may be configured to be substantially flat rather than round.

In some embodiments, at least one (e.g., both) of the first impermeable layer 601 or second impermeable layer includes a distal portion 609 that extends distally from the pocket portion 605 with the chamber 604 being absent from the distal portion 609. An absorbent material 620 may be disposed on the distal portion 609 to cover at least some or substantially all of the distal portion 609 at least on the side of the fluid collection device 600 that includes the opening 606. The absorbent material 620 may include any of a number of different absorbent materials, such as a non-woven polyester material, an open cell foam (e.g., polyurethane, diisocyanate, hydrophilic-treated polyethylene), cellulose fibers, non-woven or woven fibers, and/or gel sorbents (e.g., crosslinked sodium polyacrylate polymer beads/granules).

In some embodiments, the fluid collection device 600 also include the fluid permeable membrane 630. The fluid permeable membrane 630 is configured to extend across the opening 606 and the absorbent material 620 covering the distal portion 609. In some embodiments, the fluid permeable membrane 630 covers all of the front side of the fluid collection device 600, including the opening 606. The fluid permeable membrane 630 also may wrap around all of the fluid collection device 600 such that the fluid permeable membrane 630 also covers that back side (including the first layer 601) of the fluid impermeable barrier 602. The fluid permeable membrane 630 may include any of the wicking materials or fluid permeable membranes described herein. In an embodiment, the fluid permeable membrane 630 includes a polyimide material.

In use, the opening 606 may be positioned over at least the urethra of the user (and, in some embodiments, over the vulva of the user) such that fluid voided by the user is directed into through the opening 606 into the fluid permeable body 610. Fluid may flow downward in the fluid permeable body to the pocket portion 605 of the chamber 604 where the inlet 109 of the conduit 108 is positioned. Application of an at least partially negative pressure to the conduit 108 draws the fluid in the pocket portion 605 into the inlet 109 of the conduit 108 for removal from the fluid collection device 600. The distal portion 609 is positioned such that any overflow fluid or misdirected fluid is absorbed by the absorbent material 620. The configuration of the fluid collection device 600 allows fluid to flow into the internal chamber 604 or sump. The absorbent material 620 is positioned to capture and contain any overflow from the chamber 604 or misdirected fluid discharge, while the pocket portion 605 keeps lingering moisture away from the anatomy of the user.

FIGS. 7A-7D show various views of a fluid collection device 700, according to an embodiment. Specifically, FIG. 7A is a front view of the fluid collection device 700, FIG. 7B is a front view of the fluid collection device 700 of FIG. 7A with a wicking material 730 (e.g., fluid permeable membrane) removed, FIG. 7C is a front view of the fluid collection device of FIG. 7A with the wicking material 730 and an absorbent material 720 removed, and FIG. 7D is a cross-sectional view of a portion of the fluid collection device shown in FIG. 7C with the wicking material 730 and the absorbent material 720 removed. The fluid collection device 700 may include a fluid impermeable barrier 702, a porous material 710, and a conduit 108. In some embodiments, the fluid collection device 700 may include a generally oval or cassini oval shape.

The fluid impermeable barrier 702 may form an elongated shape with a proximal region and a distal region. The fluid impermeable barrier 702 may include a first impermeable layer 701, a second impermeable layer 703 opposite to the first impermeable layer 701 and defining an opening 706. A chamber 704 may be defined at least partially by the first impermeable layer 701 and the second impermeable layer 703. The chamber 704 may include a pocket portion 705 that extends distally from the opening 706 between the first impermeable layer 701 and the second impermeable layer 703. In some embodiments, the chamber 704 includes a pocket portion 705 that extends distally from the opening 706 between the first impermeable layer 701 and the second impermeable layer 703. The pocket portion 705 includes the region of the chamber 704 from the distal end of the opening 706 until the chamber 704 terminates distal to the distal end of the opening 706. Accordingly, the pocket portion 705 of the chamber 704 may be defined or bordered by the first impermeably layer 701, the second impermeable layer 703, and a terminating end of the chamber 704 where the first impermeable layer 701 and the second impermeable layer 703 are heat-sealed, adhered to one another, or otherwise secured together. In some embodiments, the pocket portion 705 extends distally from the distal end of the opening 706 to a terminating end of the chamber 704 at a length of at least about 2.5 cm, at least about 5 cm, at least about 7.5 cm, about 2 cm to about 5 cm, about 2.5 cm to about 4.5 cm, about 3.5 cm to about 5.5 cm, about 4.5 cm to about 6.5 cm, about 5.5 cm to about 7.5 cm, about 2.5 cm to about 3.5 cm, about 3.5 cm to about 4.5 cm, about 4.5 cm to about 5.5 cm, about 5.5 cm to about 6.5 cm, or about 6.5 cm to about 7.5 cm.

The opening 706 may be sized to cover at least the urethra of the user, and may be sized and positioned to cover the vulva of a user. The first impermeable layer 701 and the second impermeable layer 703 may be secured together, such as a heat seal near the edge of chamber 704 or other adhesive, ultrasonic, or radio frequency securement. The first impermeable layer 701 and the second impermeable layer 703 of the fluid impermeable barrier 702 may include any fluid impermeable material described herein. In some embodiments, the first impermeable layer 701 and the second impermeable layer 703 of the fluid impermeable barrier 702 may include a fluid impermeable polyurethane film.

The porous material 710 may be disposed in the chamber 704 such that the porous material 710 extends across the opening 706 and is positioned in the pocket portion 705 of the chamber 704. The porous material 710 may be configured to space the first impermeable layer 701 from the second impermeable layer 703 in the pocket portion 705 of the chamber 704. Turning to FIG. 7D and as described above, the porous material 710 may include one or more sheets of porous material 710 including a spacer fabric that imparts a three-dimensional property to the chamber 704 to space the first impermeable layer 701 from the second impermeable layer 703 in the chamber 704. In some embodiments, the porous material 710 includes the first layer 764 and the second layer 766. The first and second layers 764, 766 may be a woven material. The porous material 710 also may include the plurality of fibers 768 forming a layer (e.g., intermediary layer) between the first layer 764 and the second layer 766. Each of the first layer 764, the second layer 766, and the plurality of fibers 768 define a plurality of pores, thereby allowing transport of the bodily fluids and air circulation through the porous material 710. The porous material 710 may include any aspect of the porous material 710 described above in relation to the fluid collection device 200, such as the dimensions and materials provided in greater detail above.

In some embodiments, the porous material 710 may be disposed in the chamber 704 and/or the pocket portion 705 to be layered with multiple sheets of the porous material 710. In some embodiments, the porous material 710 is disposed in the chamber 704 such that a plurality of sheets of the porous material 710 are disposed in the chamber 704 between the opening 706 and the first impermeable layer 701, while multiple sheets of the porous material 710 are disposed in the pocket portion 705 of the chamber 704 between the first impermeable layer 701 and the second impermeable layer 703. The multiple sheets of the porous material 710 in the pocket portion 705 may be greater in number than the plurality of sheet of the porous material 710 between the opening 706 and the first impermeable layer 701. FIG. 7D shows an example of a fluid collection device 700 having three sheets 710a, 710b, 710c of the porous material 710 in the pocket portion 705 of the chamber 704 between the first impermeable layer 701 and the second impermeable layer 703. In this embodiment, then, two sheets of porous material 710 may be positioned between the opening 706 and the first impermeable layer 701. Other embodiments may include varying numbers of sheets of the porous material 710 in the pocket portion 705 and/or between the opening 706 and the first impermeable layer 701. For example, in one embodiment, the fluid collection device 700 includes three sheets of the porous material 710 between the opening 706 and the first impermeable layer 701, and six sheets of the porous material 710 in the pocket portion 705 between the first impermeable layer 701 and the second impermeable layer 703.

The conduit 108 extending through the proximal region of the fluid impermeable barrier 702 into the chamber 704. The inlet 109 of the conduit 108 may be disposed in the pocket portion 705 of the chamber 704. The sheets of the porous material 710 may contour around the conduit 108 such that there is substantially no space between adjacent sheets of the porous material 710 except where the conduit 108 is between the adjacent sheets of the porous material 710.

The fluid collection device 700 also may include the absorbent material 720 secured to the second impermeable layer 703 and covering at least some or substantially all of the second impermeable layer 703 such that the second impermeable layer 703 is positioned between the absorbent material 720 and the first impermeable layer 701. The absorbent material 720 may be shaped generally complementary to the second impermeable layer 703. In some embodiments, the absorbent material 720 defines an opening 726 aligned with and sized and shaped generally complementary to the opening 706 of the second impermeable layer 703. In some embodiments, the absorbent material 720 extends around (e.g., covers) at least some or substantially all of both the first impermeable layer 701 and the second impermeable layer 703. The absorbent material 720 may include any of a number of different absorbent materials, such as a non-woven polyester material, an open cell foam (e.g., polyurethane, diisocyanate, hydrophilic-treated polyethylene), cellulose fibers, non-woven or woven fibers, and/or gel sorbents (e.g., crosslinked sodium polyacrylate polymer beads/granules).

The fluid collection device 700 also may include a wicking material 730 secured to the absorbent material 720 and covering the absorbent material 720 such that the absorbent material 720 is positioned between the wicking material 730 and the second impermeable layer 703. The wicking material 730 may be sized and configured to cover at least some or substantially all of the opening 726 of the absorbent material 720 and the opening 706 of the second impermeable layer 703. In some embodiments, the wicking material 730 extends around (e.g., covers) at least some or substantially all of both the front side (including the openings 706, 726) of the device 700 and also the back side of the device 700. The wicking material 730 may include any of the wicking materials or fluid permeable membranes described herein. In an embodiment, the wicking material 730 includes a polyimide material.

In some embodiments, at least one (e.g., both) of the first impermeable layer 701 or second impermeable layer 703 includes a distal portion 709 that extends distally from the pocket portion 705 with the chamber 704 being absent from the distal portion 709. At least one (e.g., both) of the absorbent material 720 and the wicking material 730 may cover the distal portion 709.

The fluid collection device 700 is configured to be compatible with the underwear of the user. The first impermeable layer 701 and the second impermeable layer 703 (e.g., layers of film) may be sealed together to create a substantially flat chamber between the first impermeable layer 701 and the second impermeable layer 703. The porous material 710 may act as a wicking, frit spacer that keeps the chamber 704 and pocket portion 705 open. The conduit 108 may be inserted into the pocket portion underneath at least some (e.g., all) of the porous material 710 to transfer fluid (e.g., urine) from the fluid collection device using applied at least partially negative pressure. The absorbent material 720 may be positioned and configured to catch any fluid overflow from the pocket portion 705 or fluid discharged outside of the opening 706. The wicking material 730 contacts that skin or anatomy of the user, and is soft and comfortable against the skin of the user. This enables voided urine to pass through the wicking material while keeping the feeling of dryness on the skin of the user.

The low profile of the fluid collection device 700 combined with the flexibility of the fluid collection device 700 may enable concealment of the fluid collection device 700 beneath clothing while enabling fluid collection and subsequent drainage. The fluid collection device 700 is configured to accommodate variations in anatomy and placement with a large collection zone. Precise location relative to the labia of the user or insertion into the labia is not required with the fluid collection device 700. The fluid collection device 700 does not require an adhesive or gasket seal to the anatomy of the user to enable fluid collection. The fluid impermeable barrier 702 of the fluid collection device may include flat film(s) that are heat sealed together around the porous material. The absorbent material 720 may then be adhered to at least the second impermeable layer 703 and the conduit 108 inserted into the chamber 704. This assembly of the fluid collection device 700 is simpler than convention fluid collection devices, and does not require any injection molded components.

In use, the openings 706, 726 may be positioned over at least the urethra of the user (and, in some embodiments, over the vulva of the user) such that fluid voided by the user is directed into through the openings 706, 726 into the fluid permeable body porous material 710. Fluid may flow downward in the porous material 710 to the pocket portion 705 of the chamber 704 where the inlet 109 of the conduit 108 is positioned. Application of an at least partially negative pressure to the conduit 108 draws the fluid in the pocket portion 705 into the inlet 109 of the conduit 108 for removal from the fluid collection device 700. The distal portion 709 is positioned such that any overflow fluid or misdirected fluid is absorbed by the absorbent material 720.

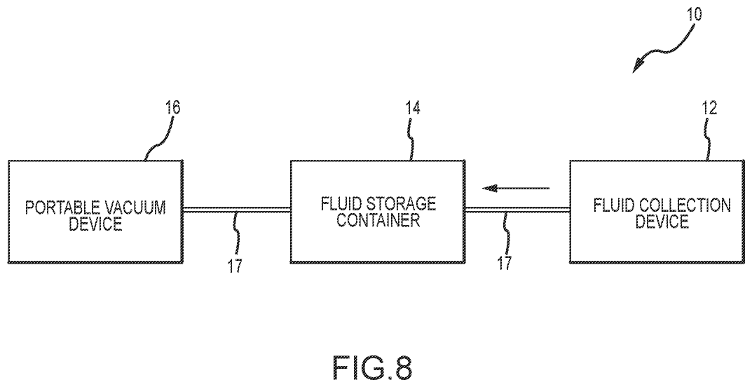

FIG. 8 is a block diagram of a system 10 for fluid collection, according to an embodiment. The system 10 may utilized with any of the fluid collection devices disclosed herein. The system 10 includes a fluid collection device 12, a fluid storage container 14, and a vacuum source 16. The fluid collection device 12 may include any of the fluid collection devices disclosed herein, such as the fluid collection device 100. The fluid collection device 12, the fluid storage container 14, and the vacuum source 16 may be fluidly coupled to each other via one or more conduits 17. The conduit 17 may include any of the conduits disclosed herein, such as the conduit 108. The fluid collection device 12 may be operably coupled to one or more of the fluid storage container 14 or the vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid storage container 14 or the vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the vacuum source 16. Accordingly, the vacuum source 16 may apply suction to the fluid collection device 12 via the fluid storage container 14. The suction force may be applied directly via the fluid storage container 14. For example, the second open end of the conduit 17 may be disposed within the vacuum source 16. An additional conduit 17 may extend from the vacuum source 16 to a point outside of the fluid collection device 12, such as to the fluid storage container 14. In such examples, the vacuum source 16 may be disposed between the fluid collection device 12 and the fluid storage container 14. In some embodiments, the vacuum source is an at least partially negative pressure source which uses a combination of negative and positive pressures to move fluid from the patient into the fluid storage container. For example, a negative pressure may be applied to the fluid collection device 12 to the inlet of the vacuum source 16, and then a positive pressure from the outlet of the vacuum source 16 to the fluid storage container.

The fluid collection device 12 may be shaped and sized to be positioned adjacent or proximate to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent or proximate to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

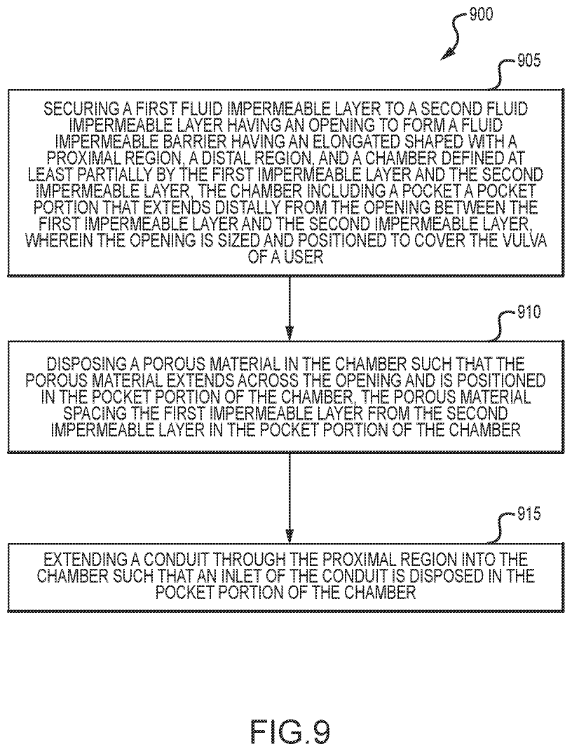

FIG. 9 is a flow diagram of a method 900 to manufacture a fluid collection device, according to an embodiment. The method 900 includes an act 905 of securing a first fluid impermeable layer to a second fluid impermeable layer having an opening to form a fluid impermeable barrier having an elongated shape with a proximal region, a distal region, and a chamber defined at least partially by the first impermeable layer and the second impermeable layer. The chamber may include a pocket portion that extends distally from the opening between the first impermeable layer and the second impermeable layer, with the opening being sized and positioned to cover the vulva of a user. The method 900 also includes an act 910 disposing a porous material in the chamber such that the porous material extends across the opening and is positioned in the pocket portion of the chamber, the porous material spacing the first impermeable layer from the second impermeable layer in the pocket portion of the chamber. The method 900 also includes an act 915 of extending a conduit through the proximal region into the chamber such that an inlet of the conduit is disposed in the pocket portion of the chamber.

In some embodiments, the method 900 further comprises securing an absorbent material to the second impermeable layer such that the absorbent material covers the second impermeable layer and an opening in the absorbent material is aligned with and generally complementary to the opening of the second impermeable layer. In some embodiments, the method 900 further comprises securing a wicking material to the absorbent material such that the wicking material covers the absorbent material and covers the opening of the absorbent material and the opening of the second impermeable layer. At least one of the first impermeable layer or second impermeable layer may include a distal portion that extends distally from the pocket portion with the chamber being absent from the distal portion, and at least one of the absorbent material and the wicking material may cover the distal portion.

In some embodiments, the method 900 further comprises securing a fluid permeable material to the second impermeable layer such that the fluid permeable material covers the second impermeable layer. At least one of the first impermeable layer or second impermeable layer may include a distal portion that extends distally from the pocket portion with the chamber being absent from the distal portion, and with the fluid permeable material covering the distal portion.

In some embodiments of the method 900, the act 910 of disposing a porous material in the chamber includes (1) disposing a plurality of sheets of the porous material in the chamber between the opening and the first impermeable layer, and (2) disposing multiple sheets of the porous material in the pocket portion of the chamber between the first impermeable layer and the second impermeable layer, the multiple sheets of the porous material being greater in number than the plurality of sheet of the porous material. In these and other embodiments, the porous material may include a first porous layer, a second porous layer, and a plurality of fibers secured to the first porous layer and the second porous layer, thereby forming an intermediary layer between the first porous layer and the second porous layer.

Acts of the method 900 are for illustrative purposes. For example, acts of the method 900 may be performed in different orders, split into multiple acts, modified, supplemented, or combined. Any of the acts of the method 900 may form various fluid collection devices disclosed herein.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A fluid collection device, comprising:
a fluid impermeable barrier having an elongated shape with a proximal region and a distal region, the fluid impermeable barrier including a first impermeable layer and a second impermeable layer opposite to the first impermeable layer and defining an opening, the first impermeable layer and the second impermeable layer at least partially defining a chamber therebetween, the chamber including a pocket portion that extends distally from the opening between the first impermeable layer and the second impermeable layer, wherein the opening is sized and positioned to cover substantially all of the vulva of a user;
a porous material disposed in the chamber such that the porous material extends across the opening and is positioned in the pocket portion of the chamber, the porous material being configured to space the first impermeable layer from the second impermeable layer in the pocket portion of the chamber; and
a conduit extending through the proximal region into the chamber and including an inlet disposed in the pocket portion of the chamber.

2. The fluid collection device of claim 1, further comprising an absorbent material secured to the second impermeable layer and covering substantially all of the second impermeable layer such that the second impermeable layer is positioned between the absorbent material and the first impermeable layer, the absorbent material including an opening aligned with and sized and shaped generally complementary to the opening of the second impermeable layer.

3. The fluid collection device of claim 2, further comprising a wicking material secured to the absorbent material and covering substantially all of the absorbent material such that the absorbent material is positioned between the wicking material and the second impermeable layer, the wicking material covering substantially all of the opening of the absorbent material and the opening of the second impermeable layer.

4. The fluid collection device of claim 3, wherein at least one of the first impermeable layer or the second impermeable layer includes a distal portion that extends distally from the pocket portion with the chamber being absent from the distal portion, at least one of the absorbent material or the wicking material covering substantially all of the distal portion.

5. The fluid collection device of claim 1, further comprising a fluid permeable material secured to the second impermeable layer and covering substantially all of the second impermeable layer such that the second impermeable layer is positioned between the fluid permeable material and the first impermeable layer, wherein at least one of the first impermeable layer or the second impermeable layer includes a distal portion that extends distally from the pocket portion with the chamber being absent from the distal portion, the fluid permeable material covering substantially all of the distal portion.

6. The fluid collection device of claim 1, wherein the porous material includes a first porous layer, a second porous layer, and a plurality of fibers secured to the first porous layer and the second porous layer, thereby forming an intermediary layer between the first porous layer and the second porous layer.

7. The fluid collection device of claim 6, wherein the porous material is disposed in the chamber such that:
a plurality of sheets of the porous material are disposed in the chamber between the opening and the first impermeable layer; and
multiple sheets of the porous material are disposed in the pocket portion of the chamber between the first impermeable layer and the second impermeable layer, the multiple sheets of the porous material being greater in number than the plurality of sheet of the porous material.

8. The fluid collection device of claim 1, wherein the porous material is disposed in the chamber such that:
a plurality of sheets of the porous material are disposed in the chamber between the opening and the first impermeable layer; and
multiple sheets of the porous material are disposed in the pocket portion of the chamber between the first impermeable layer and the second impermeable layer, the multiple sheets of the porous material being greater in number than the plurality of sheet of the porous material.

9. A fluid collection device, comprising:
a fluid impermeable barrier having an elongated shape with a proximal region and a distal region, the fluid impermeable barrier including a first impermeable layer, a second impermeable layer opposite to the first impermeable layer and defining an opening, the first impermeable layer and the second impermeable layer at least partially defining a chamber therebetween, wherein the opening is sized and positioned to cover substantially all of the vulva of a user, wherein at least one of the first impermeable layer or the second impermeable layer includes a distal portion having the chamber absent therefrom;
a porous material disposed in the chamber such that the porous material extends across the opening and is positioned in the pocket portion of the chamber;
one or more fluid permeable materials covering substantially all of the distal portion of the fluid impermeable barrier; and
a conduit extending through the proximal region into the chamber and including an inlet disposed in the chamber.

10. The fluid collection device of claim 9, wherein the one or more fluid permeable materials covering substantially all of the distal portion of the fluid impermeable barrier includes at least an absorbent material secured to the second impermeable layer and covering substantially all of the distal portion and the second impermeable layer such that the second impermeable layer is positioned between the absorbent material and the first impermeable layer, the absorbent material including an opening aligned with and sized and shaped generally complementary to the opening of the second impermeable layer.

11. The fluid collection device of claim 10, wherein the one or more fluid permeable material covering substantially all of the distal portion of the fluid permeable barrier includes the absorbent material and a wicking material secured to the absorbent material and covering substantially all of the distal portion and the absorbent material such that the absorbent material is positioned between the wicking material and the second impermeable layer, the wicking material covering substantially all of the opening of the absorbent material and the opening of the second impermeable layer.

12. The fluid collection device of claim 11, wherein the porous material includes a first porous layer, a second porous layer, and a plurality of fibers secured to the first porous layer and the second porous layer, thereby forming an intermediary layer between the first porous layer and the second porous layer.

13. The fluid collection device of claim 9, wherein the chamber includes a pocket portion that extends distally from the opening between the first impermeable layer and the second impermeable layer.

14. The fluid collection device of claim 13, wherein the porous material is disposed in the chamber such that:
a plurality of sheets of the porous material are disposed in the chamber between the opening and the first impermeable layer; and
multiple sheets of the porous material are disposed in the pocket portion of the chamber between the first impermeable layer and the second impermeable layer, the multiple sheets of the porous material being greater in number than the plurality of sheet of the porous material.

15. The fluid collection device of claim 14, wherein the porous material includes a first porous layer, a second porous layer, and a plurality of fibers secured to the first porous layer and the second porous layer, thereby forming an intermediary layer between the first porous layer and the second porous layer.

16. The fluid collection device of claim 10, wherein the porous material includes a multilayer material.

17. A method of manufacturing a fluid collection device, the method comprising:
securing a first fluid impermeable layer to a second fluid impermeable layer having an opening to form a fluid impermeable barrier having an elongated shape with a proximal region, a distal region, and a chamber defined at least partially by the first impermeable layer and the second impermeable layer, the chamber including a pocket portion that extends distally from the opening between the first impermeable layer and the second impermeable layer, wherein the opening is sized and positioned to cover substantially all of the vulva of a user;
disposing a porous material in the chamber such that the porous material extends across the opening and is positioned in the pocket portion of the chamber, the porous material spacing the first impermeable layer from the second impermeable layer in the pocket portion of the chamber; and
extending a conduit through the proximal region into the chamber such that an inlet of the conduit is disposed in the pocket portion of the chamber.

18. The method of claim 17, further comprising securing an absorbent material to the second impermeable layer such that the absorbent material covers substantially all of the second impermeable layer and an opening in the absorbent material is aligned with and generally complementary to the opening of the second impermeable layer.

19. The method of claim 18, further comprising securing a wicking material to the absorbent material such that the wicking material covers substantially all of the absorbent material and covers substantially all of the opening of the absorbent material and the opening of the second impermeable layer.

20. The method of claim 19, wherein at least one of the first impermeable layer or second impermeable layer includes a distal portion that extends distally from the pocket portion with the chamber being absent from the distal portion, at least one of the absorbent material and the wicking material covering substantially all of the distal portion.

21. The method of claim 17, further comprising securing a fluid permeable material to the second impermeable layer such that the fluid permeable material covers substantially all of the second impermeable layer, wherein at least one of the first impermeable layer or second impermeable layer includes a distal portion that extends distally from the pocket portion with the chamber being absent from the distal portion, the fluid permeable material covering substantially all of the distal portion.

22. The fluid collection device of claim 17, wherein disposing a porous material in the chamber includes:
disposing a plurality of sheets of the porous material in the chamber between the opening and the first impermeable layer; and
disposing multiple sheets of the porous material in the pocket portion of the chamber between the first impermeable layer and the second impermeable layer, the multiple sheets of the porous material being greater in number than the plurality of sheet of the porous material,
wherein the porous material includes a first porous layer, a second porous layer, and a plurality of fibers secured to the first porous layer and the second porous layer, thereby forming an intermediary layer between the first porous layer and the second porous layer.
